(19) United States
(12) Patent Application Publication
(10) Pub. No.: US 2009/0252699 A1
(43) Pub. Date: Oct. 8, 2009

(54) MEDICAL DEVICES WITH AN ANTIMICROBIAL POLYURETHANE COATING

(75) Inventors: Jurgen Kocher, Langenfeld (DE); Stefanie Eiden, Leverkusen (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/419,554

(22) Filed: Apr. 7, 2009

(30) Foreign Application Priority Data
Apr. 8, 2008 (EP) 08154207.8

(51) Int. Cl. A61K 31/74; A01N 59/16

(52) U.S. Cl. 424/78.08; 424/618; 977/773

(57) ABSTRACT

The present invention relates to a medical device comprising a coating which is obtainable starting from a solution comprising a nonionic polyurethaneurea and silver.

### RELATED APPLICATIONS

This application claims benefit to European Patent Application No. 08 154 207.8, filed Apr. 8, 2008, which is incorporated herein by reference in its entirety for all useful purposes.

### BACKGROUND OF THE INVENTION

The present invention relates to medical devices having an antibacterial (antimicrobial) polyurethane coating. Further provided by the present invention is a process for producing the medical devices with an antibacterial (antimicrobial) polyurethane coating, and also their use. The medical devices are produced preferably starting from a solution of a polyurethaneurea.

Articles made of plastic and metal are used very frequently in the medical sector. Examples of such materials are implants, cannulas or catheters. A problem associated with the use of these products is the ease with which the surfaces of these materials are colonized by microbes. The consequences of using an article colonized with bacteria, such as an implant, a cannula or a catheter, are often infections through the formation of a biofilm. Such infections are particularly serious in the area of central venous catheters and also in the urological area, where catheters are used.

Before now, in the past, numerous attempts have been made to prevent the colonization of surfaces by bacteria, and hence infections. Often it has been attempted to impregnate the surface of medical implants or catheters with antibiotics. In that case, however, account must be taken of the formation and selection of resistant bacteria.

Another approach to preventing infections when using implants or catheters is to use metals or metal alloys, in the case of catheters, for example.

Of particular significance in this context is the antibacterial effect of silver. Silver and silver salts have already been known for many years to be antimicrobially active substances. The antimicrobial effect of surfaces which contain silver derives from the release of silver ions. The advantage of silver consists in its high toxicity for bacteria, even at very low concentrations. Hardes et al., Biomaterials 28 (2007) 2869-2875, report bactericidal activity for silver at a concentration of down to 35 ppb. In contrast, even at a significantly higher concentration, silver is still not toxic to mammalian cells. A further advantage is the low tendency of bacteria to develop resistances to silver.

Various approaches at equipping medical devices with silver, such as catheters, for example, are described in the literature. One approach is the use of metallic silver on catheter surfaces. A catheter comprising a silvered surface on the outer wall is described in U.S. Pat. No. 3,800,087. A disadvantage there is that the silver adheres poorly in the face of the challenges on the catheter, such as, for example, on storage in body fluids such as urine, on friction during introduction and removal from the body, or by repeated bending of the catheter. An improvement to the adhesion of the silver coat on a catheter plastic is described by DE 4328999, by the application between plastic and silver coat of metal layers with better adhesion. In the case of the products described, the silver is applied by vapour deposition in a vacuum chamber, by sputtering or else by ion implantation. These processes are very complex and costly. A further disadvantage is that the amount of elemental silver applied by vapour deposition is relatively high, while only very small amounts of active silver ions are delivered to the surrounding fluid. Furthermore, these processes can only be used to coat the outside of an implant or a catheter. It is known, however, that bacteria also attach readily to the inside of a catheter, leading to the formation of a biofilm and the infection of the patient.

Metallic coatings on medical devices, however, not only have the disadvantage of the poor adhesion to the catheter material but also the fact that application to the insides of the catheter is very involved at the least.

Numerous applications are concerned with the use of silver salts in antimicrobial coatings which are applied to medical implants or catheters. As compared with metallic silver, silver salts have the disadvantage that in the impregnated coat, alongside the active silver, there are also anions present which under certain circumstances may be toxic, such as nitrate in silver nitrate, for example. A further problem is the rate of release of silver ions from silver salts. Certain silver salts such as silver nitrate are highly soluble in water and are therefore delivered possibly too quickly from the surface coating into the surrounding medium. Other silver salts such as silver chloride are so poorly dissolving that silver ions may be delivered too slowly to the fluid.

Further publications, exemplified by WO 2004/017738 A, WO 2001/043788 A and US 2004/0116551 A, describe a concept which involves combining different silver salts to arrive at a silver-containing coating that continuously releases silver ions. The various silver salts are mixed with different polymers, polyurethanes for example, and the combination of silver salts with different water solubilities is tailored in such a way that there is constant release of silver over the entire period in which the coated device is used. These processes, as a result of the use of a plurality of silver salts and a plurality of polymers, are complicated.

Other processes using silver ions are described by WO 2001/037670 A and US 2003/0147960 A. WO 2001/037670 A describes an antimicrobial formulation which complexes silver ions in zeolites. US 2003/0147960 A describes coatings in which silver ions are bound in a mixture of hydrophilic and hydrophobic polymers.

The processes described that use silver salts have the disadvantages mentioned before, and, moreover, are complicated to implement and therefore expensive in terms of production, and so there continues to be a need for silver-containing coatings that are improved in respect of the production process and the activity.

One interesting possibility for the antimicrobial equipping of plastics is to use nanocrystalline silver particles. The advantage to coating with metallic silver lies in the surface area of the nanocrystalline silver, which is very much greater in relation to its volume; this leads to increased release of silver ions as compared with a metallic silver coating.

Furno et al., Journal of Antimicrobial Chemotherapy 2004, 54, pp. 1019-1024, describe a process which uses supercritical carbon dioxide to impregnate nanocrystalline silver into silicone surfaces. In view of the complex impregnating operation, this process is expensive and not easy to apply.

In addition there are various known processes for incorporating nanocrystalline silver into plastics. For instance, WO 01/09229 A1, WO 2004/024205 A1, EP 0 711 113 A and Münstedt et al., Advanced Engineering Materials 2000, 2(6), pages 380 to 386 describe the incorporation of nanocrystalline silver into thermoplastic polyurethanes. Pellets of a commercially available thermoplastic polyurethane are soaked in solution with colloidal silver. To increase the antimicrobial activity, WO 2004/024205 A1 and DE 103 51 611 A1 further mention the possibility of using barium sulphate as an additive. Then, from the doped polyurethane pellets, the corresponding products, such as catheters, are produced by extrusion. This procedure, described in the publications, is disadvantageous on account of the fact that the amount of silver which remains on the polyurethane pellets after immersion is not constant and/or cannot be determined beforehand. The effective silver content of the resulting products must therefore be determined afterwards, i.e. after production of the end products. In contrast, a procedure which sets with precision the effective amount of silver to be provided in the resulting end product is not known from these publications.

A similar process is described by EP 0 433 961 A. Here again, a mixture of a thermoplastic polyurethane (Pellethane), silver powder and barium sulphate is mixed and extruded.

A disadvantage of this process is the relatively large amount of silver which is distributed throughout the plastic element. This process is therefore expensive and, as a result of the incorporation of the colloidal silver in the entire plastic matrix, the release of the silver is too slow for sufficient activity in certain cases. The improvement to the release of silver through the addition of barium sulphate represents a further, expensive work-step.

A coating solution comprising a thermoplastic polyurethane with nanocrystalline silver in an organic solvent for producing vascular prostheses is described by WO 2006/032497 A. The structure of the polyurethane is not further specified, but in view of the claiming of thermoplastics, the use of urea-free polyurethanes can be assumed. The antibacterial effect was determined by the growth of adhered Staphylococcus epidermidis cells on the surface of the test element in comparison with a control. The antibacterial action detected for the silver-containing coatings, however, can be rated as weak, since a retardation of growth by a maximum of only 33.2 h (starting from a defined threshold growth) relative to the control surface was found. For prolonged applications, as an implant or catheter, therefore, this coating formulation is unsuitable.

The publications discussed above suggest that silver is a highly interesting antimicrobial material, but that the technical proposals published for the production of antimicrobial surfaces for implants or catheters do not as yet represent a satisfactory solution.

Polyurethaneureas in organic solution are coating materials of very great interest, since they can be used to set a virtually infinite diversity of film properties. As alternatives it is also possible to prepare polyurethaneureas in aqueous dispersion. Although such purely aqueous systems do represent an alternative for certain toxicological considerations, experience shows that it is also possible to produce coatings of polyurethaneureas from organic solution without residual solvent content and hence without toxic properties which may derive from residues of the organic solvents.

Nevertheless, the coatings known from the prior art are still not satisfactory with respect to the smoothness of the surface, with respect to the strength of the coatings formed, and with respect to the release performance of the active antimicrobial substances.

It is an object of the present invention, therefore, to provide medical devices which are equipped with coatings which preferably do not have the disadvantages identified above.

The medical devices ought in particular to have a coating which preferably has a smooth surface.

Furthermore, the medical devices ought to have a coating which preferably exhibits sufficient strength.

Finally, medical devices ought to have a coating which releases the active antimicrobial substance preferably with a satisfactory performance.

### Embodiments of the Invention

An embodiment of the present invention is a medical device comprising a coating obtained from a solution comprising at least one nonionic polyurethaneurea and at least one silver-containing constituent.

Another embodiment of the present invention is the above medical device, wherein said polyurethaneurea is terminated with a copolymer unit comprising polyethylene oxide and polypropylene oxide.

Another embodiment of the present invention is the above medical device, wherein said polyurethaneurea coating is synthesized from at least the following synthesis components:
a) at least one macropolyol;
b) at least one polyisocyanate;
c) at least one diamine or amino alcohol;
d) at least one monofunctional polyoxyalkylene ether terminated with a copolymer unit comprising polyethylene oxide and polypropylene oxide; and
h) at least one silver-containing constituent.

Another embodiment of the present invention is the above medical device, wherein said at least one silver-containing constituent is selected from the group consisting of high-porosity silver powder, silver on support materials, or colloidal silver sols.

Another embodiment of the present invention is the above medical device, wherein said coating comprises nanocrystalline silver particles with an average size in the range of from 1 to 1000 nm.

Another embodiment of the present invention is the above medical device, wherein the amount of silver present in said coating, based on the amount of solid nonionic polyurethaneurea and calculated as Ag and Ag+, is in the range of from 0.1% to 10% by weight.

Yet another embodiment of the present invention is a process for producing a medical device comprising at least one coating comprising applying a solution comprising at least one nonionic polyurethaneurea and at least one silver-containing constituent to said medical device.

Another embodiment of the present invention is the above process, comprising applying said solution to said medical device by knifecoating, printing, transfer coating, spraying, spin coating, or dipping.

Yet another embodiment of the present invention is medical device comprising a coating obtained by the above process.

Yet another embodiment of the present invention is the above medical device, wherein said medical device is selected from the group consisting of contact lenses; cannulas; catheters; urological catheters; urinary catheters; ureteral catheters; central venous catheters; venous catheters; inlet catheters; outlet catheters; dilation balloons; catheters for angioplasty; catheters for biopsy; catheters for introducing a stent; catheters for introducing an embolism filter; catheters for introducing a vena cava filter; balloon catheters; expandable medical devices; endoscopes; laryngoscopes; tracheal devices; endotracheal tubes; respirators; tracheal aspiration devices; bronchoalveolar lavage catheters; catheters used in coronary angioplasty; guide rods; insertion guides; vascular plugs; pacemaker components; cochlear implants; dental implant tubes for feeding; drainage tubes; guide wires; gloves; stents; implants; extracorporeal blood lines; membranes, dialysis membranes; blood filters; devices for circulatory support; dressing materials for wound management; urine bags; stoma bags; implants which comprise a medically active agent; stents which comprise a medically active agent; balloon surfaces which comprise a medically active agent; contraceptives which comprise a medically active agent; endoscopes; laryngoscopes; and feeding tubes.

### DESCRIPTION OF THE INVENTION

This object is achieved through the provision of medical devices which have at least one coating which is obtainable starting from a solution comprising at least one nonionic polyurethaneurea and at least one silver-containing constituent.

The coating envisaged by the invention, based on a nonionic polyurethaneurea and a silver-containing constituent, is produced, then, starting from a solution of the corresponding polyurethaneurea that comprises a silver-containing constituent.

In accordance with the invention it has been found that, through the use of polyurethaneureas in organic solvents to which a silver-containing constituent has been added, it is possible to produce coatings on medical devices that are satisfactory in respect of the smoothness of the surface, in respect of the strength of the coatings formed, and in respect of the release performance of the active antimicrobial substances. Corresponding experiments in accordance with the invention, and also corresponding comparative experiments, which support this finding, are described later on below.

Polyurethaneureas for the purposes of the present invention are polymeric compounds which have
(a) repeat units containing at least two urethane groups, of the following general structure

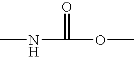

and
(b) at least one repeat unit containing urea groups

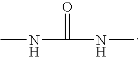

The medical devices of the invention have coatings which are based on polyurethaneureas which have substantially no ionic modification. By this is meant, in the context of the present invention, that the polyurethaneureas for use in accordance with the invention have essentially no ionic groups, such as, more particularly, no sulphonate, carboxylate, phosphate and phosphonate groups.

The term “substantially no ionic groups” or “nonionic” means, in the context of the present invention, that the resulting coatings of the polyurethaneurea contain ionic groups in a fraction of in general not more than 2.50% by weight more particularly not more than 2.00% by weight, preferably not more than 1.50% by weight, with particular preference not more than 1.00% by weight especially not more than 0.50% by weight, and even more especially no ionic groups. Hence it is particularly preferred for the polyurethaneurea not to contain any ionic groups, since high concentrations of ions in organic solution result in the polymer no longer being sufficiently soluble and hence in it being impossible to obtain stable solutions. If the polyurethane used in accordance with the invention does contain ionic groups, the groups in question are preferably carboxylates.

The polyurethaneureas provided in accordance with the invention for the coating of the medical devices are preferably substantially linear molecules, but may also be branched, though this is less preferred. By substantially linear molecules are meant systems with a slight degree of incipient crosslinking, comprising a macropolyol component as a synthesis component, preferably selected from the group consisting of a polyether polyol, a polycarbonate polyol and a polyester polyol, which have an average functionality of preferably 1.7 to 2.3, more particularly 1.8 to 2.2, more preferably 1.9 to 2.1.

If mixtures of macropolyols and, if appropriate, polyols have been used in the polyurethaneurea as elucidated in more detail below, then the functionality refers to an average value arising from the totality of the macropolyols and/or polyols.

The number-average molecular weight of the polyurethaneureas used with preference in accordance with the invention is preferably 1000 to 200 000, more preferably from 5000 to 100 000. The number-average molecular weight here is measured against polystyrene as standard in dimethylacetamide at 30° C.

### Polyurethaneureas

The polyurethaneurea-based coating systems used in accordance with the invention on the medical devices are described in more detail below.

The polyurethaneureas used in accordance with the invention in the coatings of medical devices are formed by reaction of at least one macropolyol component, at least one polyisocyanate component, preferably at least one polyoxyalkylene ether, at least one diamine and/or amino alcohol and, if desired, a polyol component. The coating on the medical devices is preferably produced starting from a solution of these polyurethaneureas.

### (a) Macropolyol Component

The composition of the polyurethaneurea provided in accordance with the invention has units which derive from at least one macropolyol component as a synthesis component.

This macropolyol component is generally selected from the group consisting of a polyether polyol, a polycarbonate polyol, a polyester polyol and any desired mixtures of these.

In one preferred embodiment of the present invention the synthesis component is formed of a polyether polyol or a polycarbonate polyol and also of mixtures of polyether polyol and a polycarbonate polyol.

In a further embodiment of the present invention the synthesis component of a macropolyol is formed of a polyether polyol, more particularly a polyether diol. Polyether polyols and, in particular, polyether diols are particularly preferred in respect of the release of silver. Corresponding experiments according to the invention that support these findings are shown later on below.

In the text below, the individual macropolyol synthesis components are described in more detail, the present invention embracing polyurethaneureas which comprise only one synthesis component, selected from, in general, polyether polyols, polyester polyols, and polycarbonate polyols, and also embracing mixtures of these synthesis components. Furthermore, the polyurethaneureas provided in accordance with the invention may also comprise one or more different representatives of these classes of synthesis components.

The above-defined functionality of the polyurethaneureas provided in accordance with the invention is understood, where two or more different macropolyols and polyols or polyamines (which are described further on below under c) and e)) are present in the polyurethaneurea, to be the average functionality.

### Polyether Polyol

The hydroxyl-containing polyethers in question are those which are prepared by polymerizing cyclic ethers such as ethylene oxide, propylene oxide, butylene oxide, tetrahydrofuran, styrene oxide or epichlorohydrin with themselves, in the presence of BF3 or of basic catalysts, for example, or by addition reaction of these ring compounds, where appropriate in a mixture or in succession, with starter components containing reactive hydrogen atoms, such as alcohols and amines or amino alcohols, e.g. water, ethylene glycol, propylene 1,2-glycol or propylene 1,3-glycol.

Preferred hydroxyl-containing polyethers are those based on ethylene oxide, propylene oxide or tetrahydrofuran or on mixtures of these cyclic ethers. Especially preferred hydroxyl-containing polyethers are those based on polymerized tetrahydrofuran. It is also possible to add other hydroxyl-containing polyethers such as those based on ethylene oxide or propylene oxide, but in that case the polyethers based on tetrahydrofuran are present at, preferably, 50% by weight at least.

### Polycarbonate Polyol

Suitable hydroxyl-containing polycarbonates are polycarbonates of a molecular weight, as determined through the OH number, of preferably 400 to 6000 g/mol, more preferably 500 to 5000 g/mol, more particularly of 600 to 3000 g/mol, which are obtainable, for example, through reaction of carbonic acid derivatives, such as diphenyl carbonate, dimethyl carbonate or phosgene, with polyols, preferably diols. Examples of suitable such diols include ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, di-, tri- or tetraethylene glycol, dipropylene glycol, polypropylene glycols, dibutylene glycol, polybutylene glycols, bisphenol A, tetrabromobisphenol A, and also lactone-modified diols.

The diol component preferably contains 40% to 100% by weight of hexanediol, preferably 1,6-hexanediol and/or hexanediol derivatives, preferably those which as well as terminal OH groups contain ether or ester groups, examples being products obtained by reaction of 1 mol of hexanediol with at least 1 mol, preferably 1 to 2 mol, of caprolactone or through etherification of hexanediol with itself to give the di- or trihexylene glycol, Polyether-polycarbonate diols as well can be used. The hydroxyl polycarbonates ought to be substantially linear. If desired, however, they may be slightly branched as a result of the incorporation of polyfunctional components, more particularly low molecular weight polyols. Examples of those suitable for this purpose include glycerol, trimethylolpropane, hexane-1,2,6-triol, butane-1,2,4-triol, trimethylolpropane, pentaerythritol, quinitol, mannitol, sorbitol, methylglycoside or 1,3,4,6-dianhydrohexitols. Preferred polycarbonates are those based on hexane-1,6-diol, and also on co-diols with a modifying action such as butane-1,4-diol, for example, or else on s-caprolactone. Further preferred polycarbonate diols are those based on mixtures of hexane-1,6-diol and butane-1,4-diol.

The polycarbonate is preferably substantially linear in construction and has only a slight three-dimensional crosslinking, with the consequence that polyurethaneureas are formed which have the specification identified above.

### Polyester Polyol

The hydroxyl-containing polyesters that are suitable are, for example, reaction products of polyhydric, preferably dihydric, alcohols with polybasic, preferably dibasic, polycarboxylic acids. In place of the free carboxylic acids it is also possible to use the corresponding polycarboxylic anhydrides or corresponding polycarboxylic esters of lower alcohols, or mixtures thereof, to prepare the polyesters.

The polycarboxylic acids may be aliphatic, cycloaliphatic, aromatic and/or heterocyclic in nature and may if appropriate be substituted, by halogen atoms, for example, and/or unsaturated. Aliphatic and cycloaliphatic dicarboxylic acids are preferred. Examples thereof include the following:

succinic acid, adipic acid, azelaic acid, sebacic acid, phthalic acid, tetrachlorophthalic acid, isophthalic acid, terephthalic acid, tetrahydrophthalic acid, hexahydrophthalic acid, cyclohexanedicarboxylic acid, itaconic acid, sebacic acid, glutaric acid, suberic acid, 2-methylsuccinic acid, 3,3-diethylglutaric acid, 2,2-dimethylsuccinic acid, maleic acid, malonic acid, fumaric acid or dimethyl terephthalate. Anhydrides of these acids can likewise be used, where they exist. Examples thereof are maleic anhydride, phthalic anhydride, tetrahydrophthalic anhydride, glutaric anhydride, hexahydrophthalic anhydride and tetrachlorophthalic anhydride.

As a polycarboxylic acid for use in small amounts, if appropriate, mention may be made here of trimellitic acid.

The polyhydric alcohols employed are preferably diols. Examples of such diols are, for example, ethylene glycol, propylene 1,2-glycol, propylene 1,3-glycol, butane-1,4-diol, butane-2,3-diol, diethylene glycol, triethylene glycol, hexane-1,6-diol, octane-1,8-diol, neopentyl glycol, 2-methyl-1,3-propanediol or neopentyl glycol hydroxypivalate. Polyester diols formed from lactones, ε-caprolactone for example, can also be employed. Examples of polyols which can be used as well if appropriate are trimethylotpropane, glycerol, erythritol, pentaerythritol, trimethylolbenzene or trishydroxyethyl isocyanurate.

### (b) Polyisocyanate

The coatings provided in accordance with the invention comprise polyurethaneureas comprising units which originate from at least one polyisocyanate as a synthesis component.

As polyisocyanates (b) it is possible to use all of the aromatic, araliphatic, aliphatic and cycloaliphatic isocyanates that are known to the skilled person and have an average NCO functionality ≧1, preferably ≧2, individually or in any desired mixtures with one another, irrespective of whether they have been prepared by phosgene or phosgene-free processes. They may also contain iminooxadiazinedione, isocyanurate, uretdione, urethane, allophanate, biuret, urea, oxadiazinetrione, oxazolidinone, acylurea and/or carbodiimide structures. The polyisocyanates may be used individually or in any desired mixtures with one another.

Preference is given to using isocyanates from the series of the aliphatic or cycloaliphatic representatives, which have a carbon backbone (without the NCO groups present) of 3 to 30, preferably 4 to 20, carbon atoms.

Particularly preferred compounds of component (b) conform to the type specified above having aliphatically and/or cycloaliphatically attached NCO groups, such as, for example, bis(isocyanatoalkyl)ethers, bis- and tris(isocyanatoalkyl)benzenes, -toluenes, and -xylenes, propane diisocyanates, butane diisocyanates, pentane diisocyanates, hexane diisocyanates (e.g. hexamethylene diisocyanate, HDI), heptane diisocyanates, octane diisocyanates, nonane diisocyanates (e.g. trimethyl-IDI (TMDI), generally as a mixture of the 2,4,4 and 2,2,4 isomers), nonane triisocyanates (e.g. 4-isocyanatomethyl-1,8-octane diisocyanate), decane diisocyanates, decane triisocyanates, undecane diisocyanates, undecane triisocyanates, dodecane diisocyanates, dodecane triisocyanates, 1,3- and 1,4-bis(isocyanatomethyl)cyclohexanes (H₆XDI), 3-isocyanatomethyl-3,5,5-trimethylcyclohexyl isocyanate (isophorone diisocyanate, IPDI), bis(4-isocyanatocyclohexyl)methane (H₁₂MDI) or bis(isocyanatomethyl)norbornane (NBDI).

Very particularly preferred compounds of component (b) are hexamethylene diisocyanate (HDI), trimethyl-HDI (TMDI), 2-methylpentane 1,5-diisocyanate (MPDI), isophorone diisocyanate (IPDI), 1,3- and 1,4-bis(isocyanatomethyl)cyclohexane (H₆XDI), bis(isocyanatomethyl)norbornane (NBDI), 3(4)-isocyanatomethyl-1-methylcyclohexyl isocyanate (IMCI) and/or 4,4′-bis(isocyanatocyclohexyl)methane (H₁₂MDI) or mixtures of these isocyanates. Further examples are derivatives of the above diisocyanates with a uretdione, isocyanurate, urethane, allophanate, biuret, iminooxadiazinedione and/or oxadiazinetrione structure and with more than two NCO groups.

The amount of constituent (b) in the polyurethaneurea provided in accordance with the invention is preferably 1.0 to 4.0 mol, more preferably 1.2 to 3.8 mol, more particularly 1.5 to 3.5 mol, based in each case on the constituent (a) of the polyurethaneurea.

### (c) Diamine or Amino Alcohol

The coatings provided in accordance with the invention comprise polyurethaneureas comprising units which originate from at least one diamine or amino alcohol as a synthesis component and as what are called chain extenders (c).

Such chain extenders are, for example, diamines or polyamines and also hydrazides, e.g. hydrazine, 1,2-ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, 1,3- and 1,4-xylylenediamine, α,α,α′,α′-tetramethyl-1,3- and -1,4-xylylenediamine and 4,4′-diaminodicyclohexylmethane, dimethylethylenediamine, hydrazine, adipic dihydrazide, 1,4-bis(aminomethyl)cyclohexane, 4,4′-diamino-3,3′-dimethyldicyclohexylmethane and other (C₁-C₄) di- and tetraalkyldicyclohexylmethanes, e.g. 4,4′-diamino-3,5-diethyl-3′,5′-diisopropyldicyclohexylmethane.

Suitable diamines or amino alcohols are generally low molecular weight diamines or amino alcohols which contain active hydrogen with differing reactivity towards NCO groups, such as compounds which as well as a primary amino group also contain secondary amino groups or which as well as an amino group (primary or secondary) also contain OH groups. Examples of such compounds are primary and secondary amines, such as 3-amino-1-methylaminopropane, 3-amino-1-ethylaminopropane, 3-amino-1-cyclohexylaminopropane, 3-amino-1-methylaminobutane, and also amino alcohols, such as N-aminoethylethanolamine, ethanolamine, 3-aminopropanol, neopentanolamine and, with particular preference, diethanolamine.

The constituent (c) of the polyurethaneurea provided in accordance with the invention can be used, in the context of the preparation of the polyurethaneurea, as a chain extender.

The amount of constituent (c) in the polyurethaneurea provided in accordance with the invention is preferably 0.05 to 3.0 mol, more preferably 0.1 to 2.0 mol, more particularly 0.2 to 1.5 mol, based in each case on the constituent (a) of the polyurethaneurea.

(d) Polyoxyalkylene ethers

The coatings provided in accordance with the invention comprise polyurethaneureas comprising preferably units which originate from a polyoxyalkylene ether as a synthesis component.

The polyoxyalkylene ether is preferably a copolymer of polyethylene oxide and polypropylene oxide. These copolymer units are present in the form of end groups in the polyurethaneurea, and have the effect of making the polyurethaneurea hydrophilic.

Suitable nonionically hydrophilicizing compounds meeting the definition of component (d) are, for example, polyoxyalkylene ethers which contain at least one hydroxyl or amino group. These polymers contain in general a fraction of 30% to 100% by weight of units derived from ethylene oxide.

Nonionically hydrophilicizing compounds (d) are, for example, monofunctional polyalkylene oxide polyether alcohols containing on average 5 to 70, preferably 7 to 55, ethylene oxide units per molecule, of the kind available in conventional manner through alkoxylation of suitable starter molecules (e.g. in Ullmanns Enzykiopädie der technischen Chemic, 4th Edition, Volume 19, Verlag Chemie, Weinheim pp. 31-38).

Examples of suitable starter molecules are saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, such as diethylene glycol monobutyl ether, for example, unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethyleyclohexylamine or dicyclohexylamine, and also heterocyclic secondary amines such as morpholine, pyrrolidine, piperidine or 1H-pyrazole. Preferred starter molecules are saturated monoalcohols. Particular preference is given to using diethylene glycol monobutyl ether as a starter molecule.

Alkylene oxides suitable for the alkoxylation reaction are, in particular, ethylene oxide and propylene oxide, which can be used in any order or else in a mixture in the alkoxylation reaction.

The polyalkylene oxide polyether alcohols are either pure polyethylene oxide polyethers or mixed polyalkylene oxide polyethers whose alkylene oxide units are composed to an extent of at least 30 mol %, preferably at least 40 mol %, of ethylene oxide units. Preferred nonionic compounds are monofunctional mixed polyalkylene oxide polyethers which contain at least 40 mol % of ethylene oxide units and not more than 60 mol % of propylene oxide units.

In accordance with the invention it has been possible to show that the polyurethaneureas with end groups based on mixed polyoxyalkylene ethers comprising polyethylene oxide and polypropylene oxide are especially suitable for producing coatings which release the active antimicrobial substance with particular efficiency. As shown later on below, this is also demonstrated experimentally.

When the alkylene oxides ethylene oxide and propylene oxide are used, they can be used in any order or else in a mixture in the alkoxylation reaction.

The average molar weight of the polyoxyalkylene ether is preferably 500 g/mol to 5000 g/mol, more preferably 1000 g/mol to 4000 g/mol, more particularly 1000 to 3000 g/mol.

The amount of constituent (d) in the polyurethaneurea provided in accordance with the invention is preferably 0.01 to 0.5 mol, more preferably 0.02 to 0.4 mol, more particularly 0.04 to 0.3 mol, based in each case on the constituent (a) of the polyurethaneurea.

### (e) Polyols

In a further embodiment the polyurethaneurea provided in accordance with the invention in the coating of the medical device further comprises units which originate from at least one polyol as a synthesis component. These polyol synthesis components, in comparison to the macropolyol, are relatively short-chain synthesis components, which through additional hard segments can give rise to stiffening.

The low molecular weight polyols (e) used to synthesize the polyurethaneureas thus have the effect, generally, of stiffening and/or branching the polymer chain. The molecular weight is preferably 62 to 500 g/mol, more preferably 62 to 400 g/mol, more particularly 62 to 200 g/mol.

Suitable polyols may contain aliphatic, alicyclic or aromatic groups. Mention may be made here, for example, of the low molecular weight polyols having up to about 20 carbon atoms per molecule, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A (2,2-bis(4-hydroxycyclohexyl)propane), and also trimethylolpropane, glycerol or pentaerythritol, and mixtures of these and, if desired, other low molecular weight polyols as well. Use may also be made of ester diols such as, for example, α-hydroxybutyl-ε-hydroxycaproic acid ester, ω-hydroxyhexyl-γ-hydroxybutyric acid ester, adipic acid β-hydroxyethyl ester or terephthalic acid bis(β-hydroxyethyl) ester.

The amount of constituent (e) in the polyurethaneurea provided in accordance with the invention is preferably 0.1 to 1.0 mol, more preferably 0.2 to 0.9 mol, more particularly 0.2 to 0.8 mol, based in each case on the constituent (a) of the polyurethaneurea.

(f) Further Amine- and/or Hydroxy-Containing Units (Synthesis Component)

The reaction of the isocyanate-containing component (b) with the hydroxy- or amine-functional compounds (a), (c), (d) and, if used, (e) takes place typically with a slight NCO excess observed over the reactive hydroxy or amine compounds. In this case, at the end point of the reaction, through attainment of a target viscosity, there still always remain residues of active isocyanate. These residues must be blocked so that there is no reaction with large polymer chains. Such a reaction leads to the three-dimensional crosslinking and gelling of the batch. The processing of such a coating solution is no longer possible, or is possible only with restrictions. The batches typically contain large amounts of alcohols. Over the course of a number of hours, on standing or on stirring of the batch at room temperature, these alcohols block the isocyanate groups that still remain.

If, however, the desire is to block the remaining, residual isocyanate content rapidly, the polyurethaneurea coatings provided in accordance with the invention may also comprise monomers (t, which are located in each case at the chain ends and cap them.

These units derive on the one hand from monofunctional compounds that are reactive with NCO groups, such as monoamines, more particularly mono-secondary amines, or monoalcohols. Mention may be made here, for example, of ethanol, n-butanol, ethylene glycol monobutyl ether, 2-ethylhexanol, 1-octanol, 1-dodecanol, 1-hexadecanol, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine and suitable substituted derivatives thereof.

Since the units (f) are used essentially in the coatings of the invention to destroy the NCO excess, the amount required is dependent essentially on the amount of the NCO excess, and cannot be specified generally.

Preferably, these units are not used during the synthesis. In that case, unreacted isocyanate is converted to terminal urethanes, preferably, by the solvent alcohols that are present in very high concentrations.

### (g) Further Constituents

Although the (antimicrobial) polyurethaneurea coating already provides the medical devices of the invention with sufficient functionalization, it may be of advantage in a specific case to integrate further functionalizations into the coating. These further possible functionalizations are now described in more detail below.

Furthermore, the polyurethaneurea coatings provided in accordance with the invention may comprise further constituents typical for the intended purpose, such as additives and fillers. An example of such are active pharmacological substances, medicaments and additives which promote the release of active pharmacological substances (drug-eluting additives).

Active pharmacological substances and medicaments which may be used in the coatings of the invention on the medical devices are in general, for example, thromboresistant agents, antibiotic agents, antitumour agents, growth hormones, antiviral agents, antiangiogenic agents, angiogenic agents, antimitotic agents, anti-inflammatory agents, cell cycle regulators, genetic agents, hormones, and also their homologues, derivatives, fragments, pharmaceutical salts, and combinations thereof.

Specific examples of such medicaments and active pharmacological substances hence include thromboresistant (non-thrombogenic) agents and other agents for suppressing acute thrombosis, stenosis or late restenosis of the arteries, examples being heparin, streptokinase, urokinase, tissue plasminogen activator, anti-thromboxan-B₂ agent; anti-B-thromboglobulin, pro staglandin-E, aspirin, dipyridimol, anti-thromboxan-A₂ agent, murine monoclonal antibody 7E3, triazolopyrimidine, ciprostene, hirudin, ticlopidine, nicorandil, etc. A growth factor can likewise be utilized as a medicament in order to suppress subintimal fibromuscular hyperplasia at the arterial stenosis site, or any other cell growth inhibitor can be utilized at the stenosis site.

The medicament or active pharmacological substance may also be composed of a vasodilatator, in order to counteract vasospasm—for example, an antispasm agent such as papaverine. The medicament may be a vaso active agent per se, such as calcium antagonists, or α- and β-adrenergic agonists or antagonists. In addition the therapeutic agent may be a biological adhesive such as cyanoacrylate in medical grade, or fibrin, which is used, for example, for bonding a tissue valve to the wall of a coronary artery.

The therapeutic agent may further be an antineoplastic agent such as 5-fluorouracil, preferably with a controlling releasing vehicle for the agent (for example, for the use of an ongoing controlled releasing antineoplastic agent at a tumour site).

The therapeutic agent may be an antibiotic, preferably in combination with a controlling releasing vehicle for ongoing release from the coating of a medical device at a localized focus of infection within the body. Similarly, the therapeutic agent may comprise steroids for the purpose of suppressing inflammation in localized tissue, or for other reasons.

Specific examples of suitable medicaments include:
(a) heparin, heparin sulphate, hirudin, hyaluronic acid, chondroitin sulphate, dermatan sulphate, keratan sulphate, lytic agents, including urokinase and streptokinase, their homologues, analogues, fragments, derivatives and pharmaceutical salts thereof,
(b) antibiotic agents such as penicillins, cephalosporins, vacomycins, aminoglycosides, quinolones, polymyxins, erythromycins; tetracyclines, chloramphenicols, clindamycins, lincomycins, sulphonamides, their homologues, analogues, derivatives, pharmaceutical salts and mixtures thereof,
(c) paclitaxel, docetaxel, immunosuppressants such as sirolimus or everolimus, alkylating agents, including mechlorethamine, chlorambucil, cyclophosphamide, melphalane and ifosfamide; antimetabolites, including methotrexate, 6-mercaptopurine, 5-fluorouracil and cytarabine; plant alkoids, including vinblastin; vincristin and etoposide; antibiotics, including doxorubicin, daunomycin, bleomycin and mitomycin; nitrosurea, including carmustine and lomustine; inorganic ions, including cisplatin; biological reaction modifiers, including interferon; angiostatins and endostatins; enzymes, including asparaginase; and hormones, including tamoxifen and flutamide, their homologues, analogues, fragments, derivatives, pharmaceutical salts and mixtures thereof; and
(d) antiviral agents such as amantadine, rimantadine, rabavirin, idoxuridine, vidarabin, trifluridine, acyclovir, ganciclovir, zidovudine, phosphonoformates, interferons, their homologues, analogues, fragments, derivatives, pharmaceutical salts and mixtures thereof; and
e) antiflammatory agents such as, for example, ibuprofen, dexamethasone or methylprednisolone.

Further typical additives and auxiliaries such as thickeners, hand assistants, pigments, dyes, matting agents, UV stabilizers, phenolic antioxidants, light stabilizers, hydrophobicizing agents and/or flow control assistants may likewise be used in the coating provided in accordance with the invention.

### (h) Antimicrobial Silver

The medical devices of the invention comprise in the corresponding coating, besides the polyurethaneurea, at least one silver-containing constituent.

By “a silver-containing constituent” is meant, for the purposes of the present invention, any component capable of releasing silver in elemental or ionic form and hence leading to an antimicrobial (biocidal/antibacterial) effect.

The biocidal effect of silver derives from the interaction of silver ions with bacteria. In order to be able to generate the maximum number of silver ions from elemental silver, a large surface area of the silver is advantageous. Consequently, for antimicrobial applications, use is made primarily of high-porosity silver powders, silver on support materials or colloidal silver sols.

Available commercially at present, for example, are Ag-Ion (silver in a zeolite, Agion, Wakefield, Mass., USA), Ionpure® (Ag+ in glass, Ciba Spezialitiätenchemie GmbH, Lampertheim, Germany), Alphasan® (AgZr phosphate, Milliken Chemical, Gent, Belgium), Irgaguard® (Ag in zeolite/glass), Hygate® (silver powder, Bio-Gate, Nuremberg, Germany), NanoSilver® BG (silver in suspension) and Nanocid® (silver on TiO2, Pars Nano Nasb Co., Tehran, Iran).

The silver powders are preferably obtained from a gas phase, a silver melt being vaporized in helium. The resultant nanoparticles agglomerate immediately and are obtained in the form of high-porosity, readily filterable powders. The disadvantage of these powders, however, is that the agglomerates can no longer be dispersed into individual particles.

Colloidal silver dispersions are obtained by reducing silver salts in organic or aqueous medium. Their preparation is more complex than that of the silver powders, but offers the advantage that unagglomerated nanoparticles are obtained. As a result of the incorporation of unagglomerated nanoparticles into coating materials it is possible to produce transparent films.

For the silver-containing polyurethaneurea coatings of the invention it is possible to use any desired silver powders or colloidal silver dispersions. A multiplicity of such silver materials is available commercially.

The silver sols used preferably to formulate the silver-containing polyurethaneurea coatings of the invention are prepared from Ag₂O by reduction with a reducing agent such as aqueous formaldehyde solution following prior addition of a dispersing assistant. For this purpose the Ag₂O sols are prepared, for example, batchwise, by rapid mixing of silver nitrate solution with NaOH, by means of rapid stirring, or in a continuous operation, by using a micromixer conforming to DE 10 2006 017 696. Thereafter the Ag₂O nanoparticles are reduced with formaldehyde in excess, in a batch process, and, finally, are purified by centrifuging or membrane filtration, preferably by membrane filtration. This mode of production is particularly advantageous on account of the fact that it allows the amount of organic auxiliaries bound on the surface of the nanoparticles to be minimized. The product is a silver sol dispersion in water with an average particle size of approximately 10 to 150 nm n, more preferably 20 to 100 nm.

For the production of antimicrobially equipped coatings it is possible to use nanocrystalline silver particles with an average size of 1 to 1000 nm, preferably 5 to 500 nm, very preferably of 10 to 250 nm. This particle size is determined by means of laser correlation spectroscopy.

The degree of crystallinity of the silver particles used is preferably 50%, more preferably 70%, very preferably 90%.

The silver nanoparticles are dispersed in organic solvents. For addition to organic solutions of polyurethaneureas, the water of the nanocrystalline silver dispersion must be replaced by an organic solvent. The silver dispersion is preferably taken up in the same solvents also used to dissolve the polyurethaneureas. Examples of such solvents are aromatic solvents such as toluene, alcohols such as ethanol or isopropanol, organic esters such as ethyl acetate or butyl acetate, and ketones such as acetone or methyl ethyl ketone. Also suitable for taking up the silver dispersion are mixtures of the stated solvents. The raw coating materials are prepared by adding the silver solutions to the polyurethane solution and then carrying out homogenization by stirring or shaking.

The amount of nanocrystalline silver, based on the amount of solid polymer and calculated as Ag and Ag+, can be varied. Typical concentrations range from 0.1% to 10%, preferably from 0.3% to 5%, more preferably from 0.5% to 3%, by weight.

In comparison to many alternative processes, the advantage of the silver-containing polyurethanes of the invention lies in the great ease of combination of the polyurethane solutions and the colloidal silver dispersions. Different silver concentrations can be set easily and precisely for different applications in accordance with requirements. Many processes of the prior art are substantially more complicated and are also not so precise in the metering of the amount of silver as the process of the invention.

In one particularly preferred embodiment the antimicrobial silver is in the form of high-porosity silver powders, silver on support materials, or in the form of colloidal silver sols, with 0.1% to 10% by weight of silver being present, based on the solid polyurethane polymer.

In a further particularly preferred embodiment the antimicrobial silver is in the form of colloidal silver sols in aqueous medium or in water-miscible organic solvents, with a particle size of 1 to 1000 nm, the amount added being 0.3% to 5% by weight, based on the solid polyurethane polymer.

In a further particularly preferred embodiment, the antimicrobial silver is in the form of colloidal silver sols in aqueous medium, with an average particle size of 1 to 500 nm, the amount added being 0.5% to 3% by weight, based on the solid polyurethane polymer.

### Polyurethaneurea Composition

In one preferred embodiment the antimicrobial coating provided in accordance with the invention on the medical device comprises a polyurethaneurea which is synthesized at least from
a) at least one macropolyol;
b) at least one polyisocyanate;
c) at least one diamine or amino alcohol; and
d) preferably at least one monofunctional polyoxyalkylene ether; and also
h) at least one antimicrobial, silver-containing constituent.

In a further embodiment of the present invention the antimicrobial coating provided in accordance with the invention on the medical device comprises a polyurethaneurea which is synthesized at least from
a) at least one macropolyol;
b) at least one polyisocyanate;
c) at least one diamine or amino alcohol;
d) at least one monofunctional polyoxyalkylene ether; and
e) at least one further polyol; and also
h) at least one antimicrobial silver-containing constituent.

In a further embodiment of the present invention the antimicrobial coating provided in accordance with the invention on the medical device comprises a polyurethaneurea which is synthesized at least from
a) at least one macropolyol;
b) at least one polyisocyanate;
c) at least one diamine or amino alcohol;
d) at least one monofunctional polyoxyalkylene ether;
e) at least one further polyol; and
f) at least one amine- or hydroxyl-containing monomer which is located at the polymer chain ends; and also
h) at least one antimicrobial silver-containing constituent.

Particularly preferred in accordance with the invention are polyurethaneurea coatings comprising a polyurethaneurea which is synthesized from
a) at least one macropolyol having an average molar weight between 400 g/mol and 6000 g/mol and a hydroxyl functionality of 1.7 to 2.3, or mixtures of such macropolyols;
b) at least one aliphatic, cycloaliphatic or aromatic polyisocyanate, or mixtures of such polyisocyanates, in an amount of 1.0 to 4.0 mol per mole of the macropolyol;
c) at least one aliphatic or cycloaliphatic diamine or at least one amino alcohol as so-called chain extender, or mixtures of such compounds, in an amount of 0.05 to 3.0 mol per mole of the macropolyol;
d) at least one monofunctional polyoxyalkylene ether, or a mixture of such polyethers, with an average molar weight between 500 g/mol and 5000 g/mol, in an amount of 0.01 to 0.5 mol per mole of the macropolyol;
e) if desired, one or more short-chain aliphatic polyols having a molar weight between 62 g/mol and 500 g/mol, in an amount of 0.1 to 1.0 mol per mole of the macropolyol; and
f) if desired, amine- or OH-containing units which are located on, and cap, the polymer chain ends; and also
h) at least one antimicrobial, silver-containing constituent.

Further preferred in accordance with the invention are polyurethaneurea coatings comprising a polyurethaneurea which is synthesized from
a) at least one macropolyol having an average molar weight between 500 g/mol and 5000 g/mol and a hydroxyl functionality of 1.8 to 2.2, or mixtures of such macropolyols;
b) at least one aliphatic, cycloaliphatic or aromatic polyisocyanate, or mixtures of such polyisocyanates, in an amount of 1.2 to 3.8 mol per mole of the macropolyol;
c) at least one aliphatic or cycloaliphatic diamine or at least one amino alcohol as so-called chain extender, or mixtures of such compounds, in an amount of 0.1 to 2.0 mol per mole of the macropolyol;
d) at least one monofunctional polyoxyalkylene ether, or a mixture of such polyethers, with an average molar weight between 1000 g/mol and 4000 g/mol, in an amount of 0.02 to 0.4 mol per mole of the macropolyol;
e) if desired, one or more short-chain aliphatic polyols having a molar weight between 62 g/mol and 400 g/mol, in an amount of 0.2 to 0.9 mol per mole of the macropolyol; and
f) if desired, amine- or OH-containing units which are located on, and cap, the polymer chain ends; and also
h) at least one antimicrobial, silver-containing constituent.
Even further preferred in accordance with the invention are polyurethaneurea coatings comprising a polyurethaneurea which is synthesized from
a) at least one macropolyol having an average molar weight between 600 g/mol and 3000 g/mol and a hydroxyl functionality of 1.9 to 2.1, or mixtures of such macropolyols;
b) at least one aliphatic, cycloaliphatic or aromatic polyisocyanate, or mixtures of such polyisocyanates, in an amount of 10.5 to 3.5 mol per mole of the macropolyol;
c) at least one aliphatic or cycloaliphatic diamine or at least one amino alcohol as so-called chain extender, or mixtures of such compounds, in an amount of 0.2 to 1.5 mol per mole of the macropolyol;
d) at least one monofunctional polyoxyalkylene ether, or a mixture of such polyethers, with an average molar weight between 1000 g/mol and 3000 g/mol, in an amount of 0.04 to 0.3 mol per mole of the macropolyol, more particular preference being given to a mixture of polyethylene oxide and polypropylene oxide; and
e) if desired, one or more short-chain aliphatic polyols having a molar weight between 62 g/mol and 400 g/mol, in an amount of 0.2 to 0.8 mol per mole of the macropolyol;
h) at least one antimicrobial, silver-containing constituent.

The above-defined compositions of the polyurethane coatings further comprise, if present in the form of solutions, at least one organic solvent.

These organic solvents may be selected for example from the group consisting of dimethylformamide, N-methylpyrrolidone, dimethylacetamide, tetramethylurea, chlorinated solvents, aromatic solvents, ethers, esters, ketones and alcohols. In this case, more particular preference is given to aromatic solvents, ethers, esters, ketones and alcohols, and mixtures of toluene and alcohols are even further preferred.

### Medical Device

The term “medical device” is to be understood broadly in the context of the present invention. Suitable, non-limiting examples of medical devices (including instruments) are contact lenses; cannulas; catheters, for example urological catheters such as urinary catheters or ureteral catheters; central venous catheters; venous catheters or inlet or outlet catheters; dilation balloons; catheters for angioplasty and biopsy; catheters used for introducing a stent, an embolism filter or a vena cava filter; balloon catheters or other expandable medical devices; endoscopes; laryngoscopes; tracheal devices such as endotracheal tubes, respirators and other tracheal aspiration devices; bronchoalveolar lavage catheters; catheters used in coronary angioplasty; guide rods, insertion guides and the like; vascular plugs; pacemaker components; cochlear implants; dental implant tubes for feeding, drainage tubes; and guide wires.

The coating solutions of the invention may be used, furthermore, for producing protective coatings, for example for gloves, stents and other implants; external (extracorporeal) blood lines (blood-carrying pipes); membranes; for example for dialysis; blood filters; devices for circulatory support; dressing material for wound management; urine bags and stoma bags. Also included are implants which comprise a medically active agent, such as medically active agents for stents or for balloon surfaces or for contraceptives.

Typically the medical device is formed from catheters, endoscopes, laryngoscopes, endotracheal tubes, feeding tubes, guide rods, stents, and other implants.

There are many materials suitable as a substrate of the surface to be coated, such as metals, textiles, ceramics or plastics, the use of plastics being preferred for the production of medical devices.

### Production of the Coatings

In the context of the present invention it is particularly preferred for the coatings of the medical devices to be produced starting from solutions of the coating composition described in more detail above.

In accordance with the invention it has emerged that the resulting coatings on medical devices differ according to whether the above-described coating composition is prepared starting from a dispersion or from a solution.

The coatings of the invention on medical devices have advantages in terms of mechanical stability when they are obtained starting from solutions of the above-described coating compositions. Moreover, the films from organic solutions are substantially smoother than those films obtained from aqueous polyurethane dispersions.

In a further embodiment the present invention therefore provides a medical device having at least one hydrophilic coating comprising at least one polyurethaneurea and a silver-containing component, the coating being produced starting from a solution of the polyurethaneurea.

The medical devices of the invention can be coated with the polyurethaneurea solutions by means of a variety of methods. Examples of suitable coating techniques for this purpose include knifecoating, printing, transfer coating, spraying, spincoating or dipping.

The organic polyurethane solutions can be prepared by any desired processes.

A procedure which has emerged as being preferred, however, is as follows:

For preparing the polyurethaneurea solutions that are used for coating in accordance with the invention, it is preferred to react with one another the macropolyol, the polyisocyanate, if appropriate, the monofunctional polyether alcohol and, if appropriate, the polyol, in the melt or in solution, until all of the hydroxyl groups have been consumed.

The stoichiometry used here between the individual components taking part in the reaction is a product of the aforementioned proportions for the coating of the invention.

The reaction takes place at a temperature of preferably between 60 and 110° C., more preferably 75 to 110° C., more particularly 90 to 110° C., temperatures of around 110° C. being preferred on account of the reaction rate. Higher temperatures may likewise be employed, but in that case there is a risk, in certain cases and as a function of the individual constituents used, that decomposition events and discolorations will occur in the resulting polymer.

In the case of the prepolymer comprising isocyanates and all of the components with hydroxyl groups, reaction in the melt is preferred, although there is a risk of excessively high viscosities in the mixtures after full reaction. In these cases it is also advisable to add solvent. However, as far as possible, not more than about 50% by weight of solvent should be present, since otherwise the dilution significantly slows the reaction rate.

In the case of the reaction of isocyanate and the components with hydroxyl groups, the reaction may take place in the melt in a period of 1 hour to 24 hours. Slight addition of amounts of solvent leads to slowing, but the reaction periods are within the same periods.

The sequence of the addition and/or reaction of the individual constituents may differ from that specified above. This may in particular be of advantage when the mechanical properties of the resulting coatings are to be modified. If, for example, all of the components containing hydroxyl groups are reacted simultaneously, the result is a mixture of hard segments and soft segments. If, for example, the low molecular weight polyol is added after the macropolyol component, defined blocks are obtained, which may bring different properties to the resultant coatings. The present invention is therefore not confined to any one sequence of addition and/or reaction of the individual constituents of the polyurethane coating.

Then further solvent is added and the chain extender diamine, in solution if desired, or the chain extender amino alcohol (compound (c)), in solution, is added.

The further addition of the solvent takes place preferably in steps, in order not to slow down the reaction unnecessarily, which in the case of complete addition of the amount of solvent would happen, for example, at the beginning of the reaction. Furthermore, in the case of a high level of solvent at the beginning of the reaction, a relatively low temperature is mandatory, and is at least co-determined by the nature of the solvent. This as well leads to a slowing of the reaction.

After the target viscosity has been attained, the residues of NCO still remaining can be blocked by a monofunctional aliphatic amine. The remaining isocyanate groups are preferably blocked by reaction with the alcohols present in the solvent mixture.

Suitable solvents for the preparation and the use of the polyurethaneurea solutions of the invention include all conceivable solvents and solvent mixtures, such as dimethylformamide, N-methylacetamide, tetramethylurea, N-methylpyrrolidone, aromatic solvents such as toluene, linear and cyclic esters, ethers, ketones and alcohols. Examples of esters and ketones are, for example, ethyl acetate, butyl acetate, acetone, γ-butyrolactone, methyl ethyl ketone and methyl isobutyl ketone.

Mixtures of alcohols with toluene are preferred. Examples of the alcohols which are used together with the toluene are ethanol, n-propanol, isopropanol and 1-methoxy-2-propanol.

In general the amount of solvent used in the reaction is such that approximately 10% to 50% strength by weight solutions, more preferably approximately 15% to 45% strength by weight solutions, more preferably approximately 20% to 40% strength by weight solutions, are obtained.

The solids content of the polyurethane solutions is generally between 5% to 60% by weight, preferably 10% to 40% by weight. For coating experiments the polyurethane solutions can be diluted arbitrarily with toluene/alcohol mixtures in order to allow the thickness of the coating to be varied. All concentrations from 1% to 60% by weight are possible; preference is given to concentrations in the 1% to 40% by weight range.

In this context it is possible to attain any desired coat thicknesses, such as, for example, from a few 100 nm up to a few 100 μm, although higher and lower thicknesses are possible in the context of the present invention.

The preparation of the silver dispersions in organic medium that are essential to the invention is accomplished by adding the silver-containing particles in solid or dispersed form to the polyurethane-polyurea solution and then carrying out homogenization by stirring or shaking.

For addition to organic polyurethane solutions, the particles are in dispersion in organic solvents.

Further additions such as, for example, antioxidants or pigments may likewise be used. It is also possible if desired, furthermore, to use further additions such as hand assistants, dyes, matting agents, UV stabilizers, light stabilizers, hydrophobicizing agents, hydrophilicizing agents and/or flow control assistants.

Starting from these solutions, then, the coatings provided in accordance with the invention are produced by the processes described above.

A wide variety of substrates can be coated in this context, such as metals, textiles, ceramics and plastics. Preference is given to coating medical devices manufactured from metals or from plastic. Examples of metals include the following: medical stainless steel or nickel titanium alloys. Many polymer materials are conceivable from which the medical device may be constructed, examples being polyamide; polystyrene; polycarbonate; polyethers; polyesters; polyvinyl acetate; natural and synthetic rubbers; block copolymers of styrene and unsaturated compounds such as ethylene, butylene and isoprene; polyethylene or copolymers of polyethylene and polypropylene; silicone; polyvinyl chloride (PVC) and polyurethanes. For better adhesion of the hydrophilic polyurethanes to the medical device, further suitable coatings may be applied as a base before these hydrophilic coating materials are applied.

The medical devices of the invention can be coated with the hydrophilic polyurethane dispersions by means of a variety of methods. Examples of suitable coating techniques for this purpose include knifecoating, printing, transfer coating, spraying, spin coating or dipping.

The advantages of the medical devices of the invention with the hydrophilic polyurethane coatings are set out by means of comparative experiments in the following examples.

All the references described above are incorporated by reference in their entireties for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

### EXAMPLES

The NCO content of the resins described in the inventive and comparative examples was determined by titration in accordance with DIN EN ISO 11909.

The solids contents were determined in accordance with DIN-EN ISO 3251.1 g of polyurethane dispersion was dried at 115° C. to constant weight (15-20 min) using an infrared drier.

The average particle sizes of the polyurethane dispersions are measured using the High Performance Particle Sizer (HPPS 3.3) from Malvern Instruments.

Unless noted otherwise, amounts indicated in % are % by weight and relate to the overall solution obtained.

### Substances and Abbreviations Used:

Desmophen® C2200: Polycarbonate polyol, OH number 56 mg KOH/g, number-average molecular weight 2000 g/mol (Bayer, A G, Leverkusen, D E)
PolyTHF® 1000: Polytetramethylene glycol polyol, OH number 110 mg KOH/g, number-average molecular weight 1000 g/mol (BASF A G, Ludwigshafen, D E)
PolyTHF® 2000: Polytetramethylene glycol polyol, OH number 56 mg KOH/g, number-average molecular weight 2000 g/mol (BASF A G, Ludwigshafen, D E)
Polyether LB 25: (monofunctional polyether based on ethylene oxide/propylene oxide, number-average molecular weight 2250 g/mol, OH number 25 mg KOH/g (Bayer A G, Leverkusen, D E)

### Example 1

This example describes the preparation of an inventive polyurethaneurea solution.

197.4 g of PolyTHF® 2000, 15.0 g of LB 25 and 47.8 g of 4,4′-bis(isocyanatocyclohexyl)methane (H₁₂MDI) were reacted at 110° C. to a constant NCO content of 2.5%. The reaction product was left to cool and was diluted with 350.0 g of toluene and 200 g of isopropanol. At room temperature a solution of 11.7 g of isophoronediamine in 90.0 g of 1-methoxypropan-2-ol was added. When the increase in molar weight was at an end and the desired viscosity range had been reached, stirring was continued for 4 hours in order to block the remaining isocyanate content with isopropanol. This gave 912 g of a 30.0% strength solution of polyurethaneurea in toluene/isopropanol/1-methoxypropan-2-ol, with a viscosity of 26 800 mPas at 22° C.

### Example 2

This example describes the preparation of an inventive polyurethaneurea solution.

194.0 g of PolyTHF® 2000, 22.6 g of LB 25 and 47.8 g of 4,4′-bis(isocyanatocyclohexyl)methane (H₁₂MDI) were reacted at 110° C. to a constant NCO content of 2.3%. The reaction product was left to cool and was diluted with 350.0 g of toluene and 200 g of isopropanol. At room temperature a solution of 12.1 g of isophoronediamine in 89.0 g of 1-methoxypropan-2-ol was added. When the increase in molar weight was at an end and the desired viscosity range had been reached, stirring was continued for 4 hours in order to block the remaining isocyanate content with isopropanol. This gave 916 g of a 30.7% strength solution of polyurethaneurea in toluene/isopropanol/t-methoxypropan-2-ol, with a viscosity of 15 200 mPas at 22° C.

### Example 3

This example describes the preparation of an inventive polyurethaneurea solution.

190.6 g of PolyTHF® 2000, 30.0 g of LB 25 and 47.8 g of 4,4′-bis(isocyanatocyclohexyl)methane (H₁₂MDI) were reacted at 110° C. to a constant NCO content of 2.3%. The reaction product was left to cool and was diluted with 350.0 g of toluene and 200 g of isopropanol. At room temperature a solution of 11.6 g of isophoronediamine in 89.0 g of 1-methoxypropan-2-ol was added. When the increase in molar weight was at an end and the desired viscosity range had been reached, stirring was continued for 4 hours in order to block the remaining isocyanate content with isopropanol. This gave 919 g of a 30.7% strength solution of polyurethaneurea in toluene/isopropanol/1-methoxypropan-2-ol, with a viscosity of 21 000 mPas at 22° C.

### Example 4

This example describes the preparation of an inventive polyurethaneurea solution.

202.2 g of Desmophen® C2200, 15.0 g of LB 25 and 47.8 g of 4,4′-bis(isocyanatocyclohexyl)methane (H₁₂MDI) were reacted at 110° C. to a constant NCO content of 2.5%. The reaction product was left to cool and was diluted with 350.0 g of toluene and 200 g of isopropanol. At room temperature a solution of 12.4 g of isophoronediamine in 94.0 g of 1-methoxypropan-2-ol was added. When the increase in molar weight was at an end and the desired viscosity range had been reached, stirring was continued for 3.5 hours in order to block the remaining isocyanate content with isopropanol. This gave 921 g of a 30.0% strength solution of polyurethaneurea in toluene/isopropanol/1-methoxypropan-2-ol, with a viscosity of 34 600 mPas at 22° C.

### Example 5

This example describes the preparation of an inventive polyurethaneurea solution.

198.6 g of Desmophen® C2200, 23.0 g of LB 25 and 47.8 g of 4,4′-bis(isocyanatocyclohexyl)methane (H₁₂MDI) were reacted at 110° C. to a constant NCO content of 2.4%. The reaction product was left to cool and was diluted with 350.0 g of toluene and 200 g of isopropanol. At room temperature a solution of 12.5 g of isophoronediamine in 95.0 g of 1-methoxypropan-2-ol was added. When the increase in molar weight was at an end and the desired viscosity range had been reached, stirring was continued for 4 hours in order to block the remaining isocyanate content with isopropanol. This gave 927 g of a 30.4% strength solution of polyurethaneurea in toluene/isopropanol/1-methoxypropan-2-ol, with a viscosity of 19 600 mPas at 22° C.

### Example 6

This example describes the preparation of an inventive polyurethaneurea solution.

195.4 g of Desmophen® C2200, 30.0 g of LB 25 and 47.8 g of 4,4′-bis(isocyanatocyclohexyl)methane (H₁₂MDI) were reacted at 110° C. to a constant NCO content of 2.3%. The reaction product was left to cool and was diluted with 350.0 g of toluene and 200 g of isopropanol. At room temperature a solution of 12.7 g of isophoronediamine in 94.0 g of 1-methoxypropan-2-ol was added. When the increase in molar weight was at an end and the desired viscosity range had been reached, stirring was continued for 4 hours in order to block the remaining isocyanate content with isopropanol. This gave 930 g of a 30.7% strength solution of polyurethaneurea in toluene/isopropanol/1-methoxypropan-2-ol, with a viscosity of 38 600 mPas at 22° C.

### Example 7

This example describes the preparation of an inventive polyurethaneurea solution.

215.0 g of PolyTHF® 2000 and 47.8 g of 4,4′-bis(isocyanatocyclohexyl)methane (H₁₂MDI) were reacted at 10° C. to a constant NCO content of 2.4%. The reaction product was left to cool and was diluted with 350.0 g of toluene and 200 g of isopropanol. At room temperature a solution of 10.0 g of isophoronediamine in 82.0 g of 1-methoxypropan-2-ol was added. When the increase in molar weight was at an end and the desired viscosity range had been reached, stirring was continued for 4 hours in order to block the remaining isocyanate content with isopropanol. This gave 905 g of a 30.2% strength solution of polyurethaneurea in toluene/isopropanol/1-methoxypropan-2-ol with a viscosity of 19 800 mPas at 22° C.

### Example 8

This example describes the preparation of an inventive polyurethaneurea solution.

107.0 g of PolyTHF® 1000 and 47.8 g of 4,4′-bis(isocyanatocyclohexyl)methane (H₁₂MDI) were reacted at 110° C. to a constant NCO content of 3.9%. The reaction product was left to cool and was diluted with 350.0 g of toluene and 200 g of isopropanol. At room temperature a solution of 10.9 g of isophoronediamine in 94 g of 1-methoxypropan-2-ol was added. When the increase in molar weight was at an end and the desired viscosity range had been reached, stirring was continued for 1.5 hours in order to block the remaining isocyanate content with isopropanol. This gave 810 g of a 20.5% strength solution of polyurethaneurea in toluene/isopropanol/1-methoxypropan-2-ol, with a viscosity of 40 000 mPas at 22° C.

### Example 9

This example describes the preparation of an inventive polyurethaneurea solution.

219.0 g of Desmophen® C2200 and 47.8 g of 4,4′-bis(isocyanatocyclohexyl)methane (H₁₂MID) were reacted at 110° C. to a constant NCO content of 2.4%. The reaction product was left to cool and was diluted with 350.0 g of toluene and 200 g of isopropanol. At room temperature a solution of 10.7 g of isophoronediamine in 90.0 g of 1-methoxypropan-2-ol was added. When the increase in molar weight was at an end and the desired viscosity range had been reached, stirring was continued for 4 hours in order to block the remaining isocyanate content with isopropanol. This gave 918 g of a 31.1% strength solution of polyurethaneurea in toluene/isopropanol/1-methoxypropan-2-ol, with a viscosity of 19 400 mPas at 22° C.

### Example 10

### Silver-Containing Polyurethane Solutions

A 0.054 molar silver nitrate solution was admixed with a mixture of 0.054 molar sodium hydroxide solution and the dispersing assistant Disperbyk 190 (manufacturer: BYK Chemie) (1 g/l) in a volume ratio of 1:1 and the mixture was stirred for 10 minutes. A brown Ag₂O nanosol was formed. Added to this reaction mixture with stirring was an aqueous 4.6 molar formaldehyde solution, and so the molar ratio of Ag+ to reducing agent was 1:10. This mixture was heated to 60° C., held at that temperature for 30 minutes and then cooled. The particles were purified by centrifugation (60 minutes at 30 000 rpm) and redispersed in fully demineralized water by introduction of ultrasound (1 minute). This operation was repeated twice. A colloidally stable sol with a solids content of 5% by weight (silver particles and dispersing assistant) was obtained in this way. The yield is just under 100%. After centrifugation, according to elemental analysis, the silver dispersion contains 3% by weight of Disperbyk 190, based on the silver content. An analysis by means of laser correlation spectroscopy showed an effective diameter for the particles of 73 nm.

For the silver sol to be added to the polyurethaneurea solutions of the invention, the silver obtained must first be redispersed in an organic medium. For this purpose the aqueous silver sol is evaporated almost to dryness on a rotary evaporator. Subsequently the silver powder is taken up in a 2.1 toluene/isopropanol mixture and redispersed for a few seconds using an ultrasound probe.

50 ml of the polyurethane solutions from Examples 1 to 9 were admixed with a 15% colloidal silver dispersion, whose preparation is described above, and the mixtures were homogenized by shaking. The amount of silver dispersion added to the polyurethane solutions of Examples 1 to 9 is such that the dispersions contain 1% by weight of silver, based on the solid polymer content.

TABLE 1
Polyurethane solutions with 1% by
weight of nanocrystalline silver

| Example | Product |
|---|---|
| 10a | PU solutions of Example 1 with 1% by weight |
| | nanocrystalline silver |
| 10b | PU solutions of Example 2 with 1% by weight |
| | nanocrystalline silver |
| 10c | PU solutions of Example 3 with 1% by weight |
| | nanocrystalline silver |
| 10d | PU solutions of Example 4 with 1% by weight |
| | nanocrystalline silver |
| 10e | PU solutions of Example 5 with 1% by weight |
| | nanocrystalline silver |
| 10f | PU solutions of Example 6 with 1% by weight |
| | nanocrystalline silver |
| 10g | PU solutions of Example 7 with 1% by weight |
| | nanocrystalline silver |
| 10h | PU solutions of Example 8 with 1% by weight |
| | nanocrystalline silver |
| 10i | PU solutions of Example 9 with 1% by weight |
| | nanocrystalline silver |

### Example 11

### Ag Release Study, Chemical

The silver-containing coatings for the measurement of silver release were produced on plaques made of polyurethane (thermoplastic polyurethane Texin 3041, Bayer MaterialScience AG) and measuring 25×75 mm with the aid of a spincoater (RC5 Gyrset 5, Karl Süss, Garching, Germany). For this purpose the plaques were clamped on the sample plate of the spincoater and covered homogeneously with about 2.5-3 g of the polyurethane solutions. For this purpose the polyurethane solutions used, from Examples 1-9, were diluted to half the original concentration with a mixture of toluene and isopropanol (65% by weight/35% by weight). Rotation of the sample plate at 1300 revolutions per minute for 20 seconds gave homogeneous coatings, which were dried at 50° C. for 24 hours. From the coated plaques thus obtained, sections of around 4 cm2 were produced and were used for measuring the amounts of silver released.

The various silver-containing polyurethane coatings of Examples 10a to 10i were covered with 2.5 ml of distilled water in a tablet tube and stored in an incubator at 37° C. for one week. The water was removed and the amount of silver delivered from the film to the liquid was determined by atomic absorption spectroscopy. The dry films on the polyurethane plaques sections were again covered with 2.5 ml of water and stored further at 37° C. The entire process was repeated 5 times, allowing silver release to be determined for a number of weeks.

TABLE 2
Silver release of the films in an aqueous environment

| | Ex. | Ex. | Ex. | Ex. | Ex. | Ex. | Ex. | Ex. | Ex. |
|---|---|---|---|---|---|---|---|---|---|
| | 10a | 10b | 10c | 10d | 10e | 10f | 10g | 10h | 10i |
| ng Ag | 70 | 128 | 145 | 148 | 53 | 70 | 25 | 58 | 50 |
| (Week 1) | | | | | | | | | |
| ng Ag | 33 | 70 | 55 | 13 | 8 | 5 | 23 | 38 | 15 |
| (Week 2) | | | | | | | | | |
| ng Ag | 58 | 163 | 113 | 15 | 83 | 40 | 68 | 115 | 25 |
| (Week 3) | | | | | | | | | |
| ng Ag | 15 | 28 | 18 | 5 | 5 | 5 | 3 | 15 | 5 |
| (Week 4) | | | | | | | | | |
| ng Ag | 15 | 28 | 30 | 8 | 5 | 13 | — | 20 | 5 |
| (Week 5) | | | | | | | | | |

The results show that the coatings deliver silver over a relatively long period of time. Higher levels of silver release are produced by the coatings 10a to 10f, equipped with Polyether LB 25. Over a period of 5 weeks, the films 10b and 10c, produced with PolyTHF® 2000 and LB 25, release the somewhat higher levels of silver than the films 10d to 10i produced with polycarbonate.

### Example 12

### Antimicrobial Activity of Silver-Containing Coatings

Polyurethane plaques comprising the silver-containing polyurethane dispersions of Examples 10a to 10i were investigated for their bactericidal action in a bacterial suspension of Escherichia coli ATCC 25922.

The test microbe, E. coli ATCC 25922, was cultured in an overnight culture on Columbia agar (Columbian blood agar plates, Becton Dickinson, #254071) at 37° C. Thereafter a number of colonies were suspended in PBS (PBS pH 7.2, Gibco, 920012) with 5% Müller Hinton medium (Becton Dickinson, #257092) and a cell count of approximately 1×10⁵ microbes/ml was set. 100 μl of each of these suspensions was dispersed over the test material using a piece of Parafilm measuring 20×20 mm, so that the surface was wetted uniformly with cell suspension. Thereafter the test material with the bacterial suspension was incubated in a humidity chamber at 37° C. for 6 hours. After 6 hours, 20 μl of the cell suspension were taken for the purpose of monitoring growth. The cell count was determined by carrying out serial dilution and plating out the dilution stages on agar plates. Only living cells were determined. The cell count was reported in the form of colony-forming units (CFU)/ml. Subsequently the Parafilm was removed from the test material, and the test material was washed with 3 times 4 ml of PBS in order to remove free-floating cells.

For determining the anti-adhesive activity, the coated plaques were washed with three times 4 ml of PBS in order to remove cells that had not adhered. Thereafter the coated plaques were transferred to 15 ml of PBS and sonicated in an ultrasound bath for two minutes in order to detach the adhering cells. The PBS solution containing the detached cells was likewise analysed for its cell count, by dilution series and plating out on agar plates. Here again, only living cells were counted. If there was sufficient test material available, this test was carried out in each case three times, independently of one another. The result was reported as the average value in CFU/ml with standard deviation.

TABLE 3
Antibacterial action of the coatings of Examples 10a to 10i

| Coating | Microbial growth (CFU/ml) | Cell adhesion (CFU/ml) |
|---|---|---|
| Example 10 a | <10³ | <10² |
| Example 10 b | 8 × 10³ | <10² |
| Example 10 c | <10³ | <10² |
| Example 10 d | <10³ | <10² |
| Example 10 e | <10³ | <10² |
| Example 10 f | 1 × 10⁴ | <10² |
| Example 10 g | <10³ | <10² |
| Example 10 h | 1 × 10⁸ | <10² |
| Example 10 i | 1 × 10⁷ | 5 × 10² |
| Negative control | 1 × 10⁸ | 5 × 10⁵ |
| (glass slide) | | |

The table illustrates very clearly the antibacterial activity of the silver-containing coatings analysed. Apart from one material, all of the other coatings show no significant adhered cell populations on the coating, which is very important for the formation of a biofilm. Furthermore, in the case of the majority of the coatings, microbial growth in the surrounding bacterial suspensions is suppressed significantly as well.

1. A medical device comprising a coating obtained from a solution comprising at least one nonionic polyurethaneurea and at least one silver-containing constituent.

2. The medical device of claim 1, wherein said polyurethaneurea is terminated with a copolymer unit comprising polyethylene oxide and polypropylene oxide.

3. The medical device of claim 1, wherein said polyurethaneurea coating is synthesized from at least the following synthesis components:
a) at least one macropolyol;
b) at least one polyisocyanate;
c) at least one diamine or amino alcohol;
d) at least one monofunctional polyoxyalkylene ether terminated with a copolymer unit comprising polyethylene oxide and polypropylene oxide; and
h) at least one silver-containing constituent.

4. The medical device of claim 1, wherein said at least one silver-containing constituent is selected from the group consisting of high-porosity silver powder, silver on support materials, or colloidal silver sols.

5. The medical device of claim 1, wherein said coating comprises nanocrystalline silver particles with an average size in the range of from 1 to 1000 nm.

6. The medical device of claim 1, wherein the amount of silver present in said coating, based on the amount of solid nonionic polyurethaneurea and calculated as Ag and Ag+, is in the range of from 0.1% to 10% by weight.

7. A process for producing a medical device comprising at least one coating comprising applying a solution comprising at least one nonionic polyurethaneurea and at least one silver-containing constituent to said medical device.

8. The process of claim 7, comprising applying said solution to said medical device by knifecoating, printing, transfer coating, spraying, spin coating, or dipping.

9. A medical device comprising a coating obtained by the process of claim 7.

10. The medical device of claim 1, wherein said medical device is selected from the group consisting of contact lenses; cannulas; catheters; urological catheters; urinary catheters; ureteral catheters; central venous catheters; venous catheters; inlet catheters; outlet catheters; dilation balloons; catheters for angioplasty; catheters for biopsy; catheters for introducing a stent; catheters for introducing an embolism filter; catheters for introducing a vena cava filter; balloon catheters; expandable medical devices; endoscopes; laryngoscopes; tracheal devices; endotracheal tubes; respirators; tracheal aspiration devices; bronchoalveolar lavage catheters; catheters used in coronary angioplasty; guide rods; insertion guides; vascular plugs; pacemaker components; cochlear implants; dental implant tubes for feeding; drainage tubes; guide wires; gloves; stents; implants; extracorporeal blood lines; membranes, dialysis membranes; blood filters; devices for circulatory support; dressing materials for wound management; urine bags; stoma bags; implants which comprise a medically active agent; stents which comprise a medically active agent; balloon surfaces which comprise a medically active agent; contraceptives which comprise a medically active agent; endoscopes; laryngoscopes; and feeding tubes.
